(19) United States
(12) Patent Application Publication
(10) Pub. No.: US 2023/0096884 A1
(43) Pub. Date: Mar. 30, 2023

(54) PORTABLE MEDICAL DEVICE

(71) Applicant: SUPERSONIC IMAGINE, Aix-en-Provence (FR)

(72) Inventor: Damien LERAT, Aix-en-Provence (FR)

(73) Assignee: SUPERSONIC IMAGINE, Aix-en-Provence (FR)

(21) Appl. No.: 17/956,222

(22) Filed: Sep. 29, 2022

(30) Foreign Application Priority Data
Sep. 29, 2021 (FR) 2110272

(51) Int. Cl. A61B 8/00

(52) U.S. Cl. CPC A61B 8/4427; A61B 8/4254; A61B 8/4218; A61B 8/462; A61B 2560/0431

(57) ABSTRACT

This disclosure relates to a portable medical device comprising:

a medical equipment for the acquisition of data and a carrier system, wherein the medical equipment comprises:
a processing unit configured to process the data acquired using a probe linked to a processing unit,
and wherein the carrier system is configured to equip the body of the user of the device with the medical equipment.

### TECHNICAL FIELD

This invention relates to medical systems and procedures using such systems. A system of this kind may constitute a medical examination system. More specifically, it may constitute an ultrasound device.

### PRIOR ART

A medical visualization system usually includes electronic means, for example, a sensor and/or a probe (e.g., an ultrasound probe) to acquire the patient's data, and a processor for handling the data acquired. It may also include a steering module to steer the system linked, in particular, to the user interface. The medical device may be intended to provide the data on a medium to be examined, which is displayed on a screen. In the event of medical applications, the medium is a body, for instance, a part of a patient's body (muscles, fetus, chest, liver, abdomen, etc.). The probe is generally held against the surface of a medium being examined in order to acquire data about this medium.

Often, the user of the medical device is looking at a screen on the device at the same time. However, the screen is not generally directly adjacent to the medium. For example, in the event where a patient is examined lying on a bed or sitting in a chair, the screen is often placed on a cart (or, more generally, a platform) placed adjacent to the bed or the chair and, therefore, (from the point of view of the user) in a completely different direction to that of the medium. As a result, the user of the medical device is forced to adopt an uncomfortable and unnatural position while acquiring the data. The user is in particular forced to turn their head and generally also their entire torso.

In this context, international literature shows that 80% to 90% of sonographers experience pain while carrying out scans at one point or another in the course of their career. About 20% of them suffer a career- or life-changing injury.

There are various types of medical devices available. A medical device, for example, a classical ultrasound device, is available in the above-mentioned form on an ultrasound cart on wheels (or, more generally, on an ultrasound platform), which is configured to be moved around the floor. The cart is configured to support the relatively heavy parts of the medical device, in particular, the processing unit or the screen device(s). Devices of this type may have a relatively good image quality and be versatile (due to the possibility of using several different types of probe), but they are hard to maneuver. These types of device also generally need a main power source.

Fully integrating the medical device into the examination chair or bed has also been proposed. However, such systems are generally relatively heavy and they are even more difficult to maneuver.

There are also portable ultrasound devices that use, for instance, a smartphone or an electronic tablet for imaging. In this case, the probe communicates with the smartphone or the tablet. Such systems can be easily moved, but they generally contain only a single probe. Furthermore, they have a relatively small screen and limited self-sufficiency as their batteries are relatively small. Due to the relatively compact and light nature of the parts of such a system and the reduced self-sufficiency, the image quality is generally also lower than that of a classic ultrasound device. They also always have to be held in the user's hands, preventing the user from using their hands for other tasks. This may also spread hospital-acquired infections.

### INVENTION DISCLOSURE

The purpose of this disclosure is, therefore, to provide a portable medical device that remedies the above-mentioned disadvantages. In particular, the aim is to provide a portable medical device with optimized flexibility, self-sufficiency, and versatility. It is also desirable for the medical device to permit the user of the device to avoid uncomfortable and/or unnatural positions while acquiring the data and to free their hands from holding the device.

This disclosure relates to a portable medical device, comprising:

a medical equipment for the acquisition of data and a carrier system, wherein the medical equipment comprises:

a processing unit configured to process the data acquired using a probe linked to a processing unit,

and wherein the carrier system is configured to equip the body of the user of the device with the medical equipment.

By providing a medical device of this type, the user is able to wear (or equip themselves with) the carrier system, enabling a high degree of maneuverability on the part of the user. In other words, the user can, for example, move around while always having the medical equipment with them. In this context, different elements of the medical equipment (described below) may, for instance, be arranged on the medical device in such a way that they are always close at hand for the user regardless of their position. In this way, the user is not obliged to adopt uncomfortable positions to view the information during the examination, and the elements needed for the examination (such as a probe) may be easily accessed at all times. Since these elements may be held by the carrier system, the user can have their hands free when they are not handling the elements. The carrier system can also help support the weight of the medical equipment.

The carrier system may be configured to be worn and/or placed over the shoulders of the user of the device.

The medical device may be an ultrasound device. The medical equipment for acquiring data may also be ultrasound equipment and/or an ultrasound probe.

The medical equipment may also comprise the probe. The probe may be an ultrasound probe.

The processing unit may comprise a battery and/or may be configured to function independently powered by an external electrical supply.

The medical device may further comprise a control unit with a user interface configured to enable the user to control the medical device.

The processing unit may be configured to receive and/or process the external data from an external system. For example, such external data may comprise medical data acquired by another device, for example, magnetic resonance imaging (MRI) data and/or computerized tomography (CT) data, and/or data acquired by another ultrasound device. The processing unit may comprise a communication interface, for example, a wireless one, to receive external data and/or to send the processed data to/from an external system.

The medical equipment may also comprise a display screen and/or a headset, such as an augmented reality headset. The processing unit may be configured to ensure that the screen device and/or the headset displays information based on the processed data and/or the external data.

The carrier system may be configured to be adjusted to the user's body and/or to be installed against/on the user's body.

The carrier system may comprise a backpack configured to carry the processing unit.

The backpack may comprise a belt system configured to be worn on the user's torso and to use the user's torso to support at least some of the weight of the carrier system and/or the medical equipment.

The backpack and/or the belt system may be configured to carry the battery and/or the control unit, and/or the accessories configured to enable their use in preparation for and/or during a medical examination. These accessories may, for example, comprise an identifier code reader (e.g., to identify a marker or a biopsy kit), protection for the medical equipment, sensors, and/or gloves. The identifier code reader may, for example, be a barcode reader, a QR code reader, or an RFID chip reader.

The carrier system may comprise a first arm configured to hold the probe (and/or one or more accessories) in a position such that the probe may be applied by the user's hand.

The first arm may comprise a probe holder configured to hold the probe.

The first arm may further be configured to hold and/or contain a cable connecting the probe to the processing unit.

The cable may be extended outside of the backpack and/or outside of the first arm.

At least one element of the first arm, the second arm, and the probe holder may be motorized.

The processing unit may be configured to automatically control the movement of the first and/or second arm and/or of the probe holder.

The first arm and/or the probe holder may comprise at least one localization sensor.

The processing unit may be configured to determine a movement of the probe based on the data acquired by the localization sensor(s).

The processing unit may also be configured to merge the data acquired by the probe with external data, in particular based on the data acquired by the localization sensor.

Finally, at least one element of the first arm, the second arm, and the processing unit may be attached in a reversible manner to the carrier system in order to adapt to the user's dominant hand (whether the user is left-handed or right-handed).

The first arm may be mobile and/or flexible, ensuring that the position adopted by the probe is adjustable.

The first arm may be mobile, ensuring that the probe may be shifted from the extended position to a retracted position in which the probe may be closer and/or adjacent to at least one element of the user's body, the backpack, and/or the belt system.

The carrier system may comprise a second arm configured to hold the screen device in an extended position relative to the user such that the information displayed may be accessible to the user and/or the patient.

The second arm may be mobile, ensuring that the position of the screen device remains adjustable.

The second arm may be mobile, ensuring that the display device may be shifted from the extended position to a retracted position in which the display device may be closer and/or adjacent to at least one element of the user's body, the backpack, and the belt system.

The first and/or the second arm may be attached in a mobile manner to the backpack and/or the belt system.

The first and/or the second arm may be configured to be held by the backpack and/or the belt system.

The carrier system may comprise one or two feet and/or legs configured to support at least part of the weight of the carrier system and/or of the medical device on a ground the user is situated on. The leg(s) may be retractable. The carrier system may also be equipped with three legs that may be used simultaneously or alternatively to ensure optimal stability for the user, in particular, if the ground has significant inclines. In this way, the leg(s) also enable procedures to be carried out in areas that are very uneven.

The at least one foot and/or leg may be configured to be articulated and/or mobile, enabling the user to move and/or walk (and/or rest from the weight of the carrier system) while the foot and/or leg supports at least some of the weight of the carrier system and/or the medical equipment on the ground.

The at least one foot and/or leg may be configured to be connected to the user's lower limbs in order to follow the movement of the user's lower limbs.

The carrier system may comprise or form an exoskeleton.

The exoskeleton may comprise at least one of the following elements: the backpack, the belt system, the first arm, the second arm, and the at least one foot and/or leg.

The characteristics and advantages of the invention will appear upon reading the description, given solely by way of non-limiting example, and with reference to the accompanying figures. In particular, the examples illustrated in the figures may be combined, unless there is any significant inconsistency.

### BRIEF DESCRIPTION OF THE FIGURES

FIG. 1a is a schematic front view of the first embodiment of the device according to this disclosure in a standing position.

FIG. 1b is a schematic lateral view of the device in FIG. 1a.

FIG. 2a is a schematic front view of the device in FIG. 1a and FIG. 1b in a seated position.

FIG. 2b is a schematic lateral view of the device in FIG. 2a.

FIG. 3a is a schematic front view of the device in FIG. 1a, FIG. 1b, FIG. 2a, and FIG. 2b in a moving and/or stationary position.

FIG. 3b is a schematic lateral view of the device in FIG. 3a.

FIG. 4 is a schematic front view of the second embodiment of the device according to this disclosure, comprising a head-mounted display (or virtual reality headset) and a user interface.

FIG. 5 is a schematic front view of the third embodiment of the device according to this disclosure, comprising accessories.

FIG. 6a is a schematic front view of the fourth embodiment of the device according to this disclosure.

FIG. 6b is a schematic lateral view of the device in FIG. 6a with optional modifications.

FIG. 7a is a schematic front view of the fifth embodiment of the device according to this disclosure.

FIG. 7b is a schematic lateral view of the device in FIG. 7a with optional modifications.

FIG. 8a is a schematic front view of the sixth embodiment of the device according to this disclosure.

FIG. 8b is a schematic lateral view of the device in FIG. 8a.

### DESCRIPTION OF THE EMBODIMENTS

Across the various figures provided for illustrative purposes, the same numerical references denote the identical or similar elements. The different embodiments demonstrated in the Figures may be combined, unless there is any significant inconsistency.

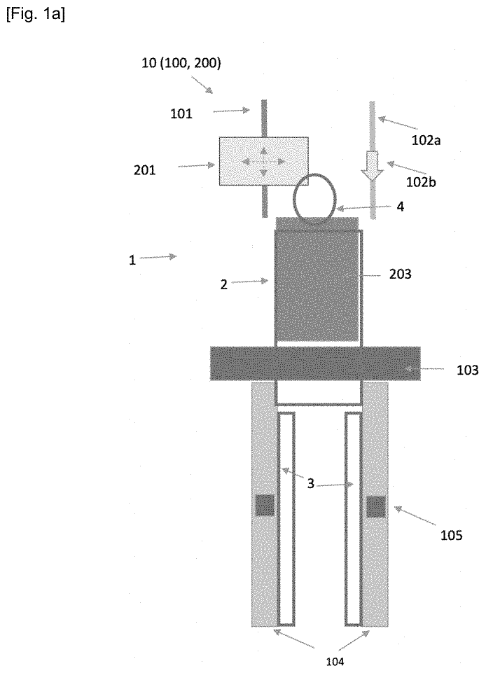

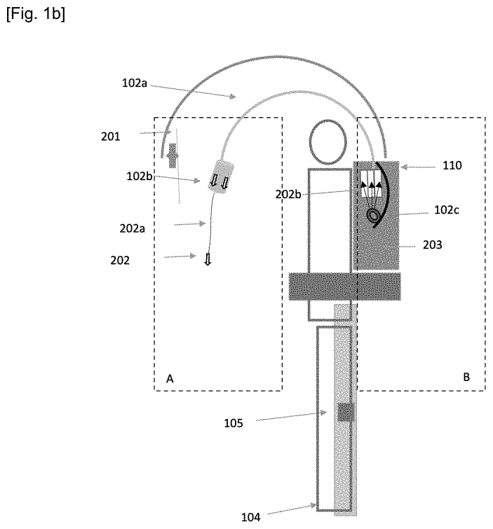

FIG. 1a is a schematic front view of the first embodiment of the device 10 according to this disclosure in a standing position. FIG. 1b is a schematic lateral view of the device in FIG. 1a. The portable ultrasound device 10 may comprise a carrier system 100 and a medical equipment 200. The medical equipment may comprise a processing unit 203 configured to process the data acquired using a probe 202 connected to a processing unit. The probe may also be part of the portable medical device 10, in particular of the medical equipment 200, or constitute an external element. The probe may be an ultrasound probe. In other words, the probe may comprise one or more ultrasound transducers.

However, the probe may also be configured to use technologies other than the ultrasound acquisition technique. For example, it may be or comprise other types of sensors or combined data acquisition techniques. In one example, the probe may comprise one or more sensors and/or optical transducers, and/or lasers. It is also possible for the probe to be configured for optical/acoustic data acquisition. As a result, the processing unit may also be configured to process types of data other than ultrasound data. Furthermore, the device may also be configured to cooperate with other types of medical devices, for example, one or more surgical devices (e.g., a cryoablation device, marker, and/or device configured to extract material from the medium, such as a biopsy needle or any surgical instrument used in combination with the device). As a result, the device 10 may be configured to comprise or be used with any combination of these probes, sensors, and medical devices.

The carrier system 100 may be configured to equip the body of the user 1 with the ultrasound equipment 200. In particular, the carrier system may be configured to adjust to the user's body and/or to be mounted on/against the user's body/torso. For instance, the carrier system 100 may be configured to be worn by the user. The carrier system 100 may in particular comprise or be available in the form of an exoskeleton. Subsequently, the user may be able to move (e.g., walk and/or sit) with the carrier system 100 on them.

For example, carrier system 100 may comprise a backpack 110 configured to carry the processing unit 203. The backpack 110 may comprise a belt system 103 configured to be worn on the user's torso and to use the user's torso to support at least some of the weight of the carrier system 100 and/or the medical equipment 200. However, the belt system 203 may also be an element of the carrier system that is distinct from the backpack 110.

In one example, the belt system may comprise a belt configured to go round the user's hips or to be worn on the user's hips. The belt system may also comprise one or more straps, for example, in the form of backpack straps.

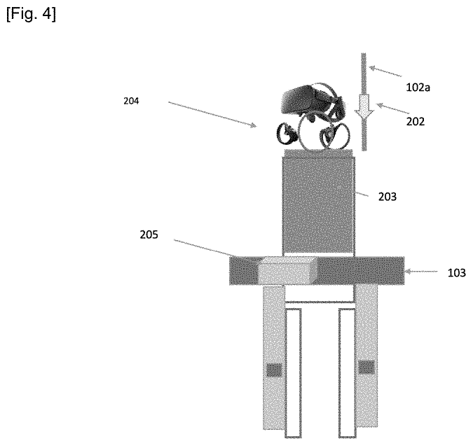

The belt system may also be configured in the manner of a harness. In this case, the belt system or the carrier system may be less cumbersome, relatively light, and individually adjusted to the user's body shape. The backpack and/or belt system may be configured to carry a battery and/or control unit with a user interface (see for example control unit 205 in FIG. 4). The control unit may enable the user or a collaborator to control the medical equipment.

The battery may be configured to supply the device 10, in particular, the medical equipment 200, with electrical energy. In this way, the mobile device 100 and in particular, its medical equipment may be configured to operate independently powered by an external electrical supply. In this way, the user can move without being hampered by any electrical supply cables.

The carrier system 100 may comprise a first arm 102a configured to hold the probe 202 in a position such that the probe may be easily and directly applied by the user's hand. For example, the first arm may be arranged in such a manner that it extends above a shoulder and/or the head of the user 1, in particular above the user's back in the direction of the front face. For example, the arm may be attached to the backpack and extend from this towards the front face of the user, where the probe is held by the first arm.

The first arm 102a may further be configured to hold and/or contain a cable 202a linking probe 202 to the processing unit 203, in particular a connector 202b of the processing unit 203. The first arm may comprise a probe holder 102b, for example, on its front end (or protruding end), which is configured to hold probe 202. The probe holder may be configured to receive and/or attach one or more probes and/or accessories.

The first arm may be mobile, ensuring that the position adopted by the probe 202 is adjustable.

The first and/or second arm may, for example, be made of a material with a certain degree of flexibility, a system of springs, or mechanical elements; it may be elastic and/or pliable (for example a plastic or a polycarbonate). At the same time, the first and/or second arm may be sufficiently rigid to support the weight of the probe and/or the cable. The first and/or second arm may also comprise one or more hinges (in particular, if the arm is made of a rigid material, such as metal).

In general, the first and/or second arm may be developed in different ways using various properties:

According to the first example, the first and/or second arm may be configured to enable elastic deformation. For instance, the first and/or second arm may comprise or be made of a material that folds and returns to its initial form, such as titanium, fiberglass, metal (steel). The elasticity may for example be caused by the structure of the arms (e.g., by a type of spring) and/or by the properties of the material.

According to the second example, the first and/or second arm may have flexible properties: for instance, the first and/or second arm may comprise or be made of steel or plastic. The flexible property may be achieved by a flexible gooseneck tube. This type of device may be controlled using a cable or cables.

According to the third example, the first and/or second arm may have rigid properties: for instance, the first and/or second arm may comprise rigid segments made of carbon, aluminum, plastic, linked by joints (balls) supported, such as actuators and/or springs. These joints may be operated using electric motors, actuator(s), such as hydraulic actuators or cables.

According to the fourth example, the first and/or second arm may have flexible properties linked to the shape memory: for example, the first and/or second arm may have shape memory alloy properties (simple or double memory).

In particular, according to the fourth example, the first and/or second arm may have the properties of a filled or hollow tube made of a shape memory alloy, the selection of which is sufficient to support the weight of the equipment. The alloy may, for example, be nitinol (nickel and titanium alloy).

Furthermore, according to the fourth example, the first and/or second arm may have the properties of a heating and cooling system adjacent to the tube or inside the tube, enabling the arm to fold into a stored position and/or to be unfolded. The percentage of nickel relative to the titanium may be selected to control the temperature at which the arm changes shape.

The examples above may also be combined on a discretionary basis. For example, a gooseneck may be used in addition to a rigid assembly in such a way as to create a digit at the end of the arm. Furthermore, the shape memory springs may be used instead of the cables used to motorize the gooseneck and/or replace the actuators in the rigid assemblies. Furthermore, the flexible materials may be used to avoid harming the user and patient and/or to make the examination more comfortable if the arm is motorized. Furthermore, the first and/or second arm and/or the probe holder may be motorized.

For example, the first arm may comprise one or more actuators (e.g., electromechanical actuators (and/or made of shape memory alloys), such as a motor) configured to automatically move the arm. The processing unit or any external control unit may be configured to control the movement of the arm. In this way, the arm may be controlled to make a specific movement, for example, the provision of the probe and/or guidance of the shifting of the probe during the data acquisition.

Furthermore, the first arm and/or the probe holder may comprise a localization sensor. In this case, the processing unit may be configured to determine the probe's shift (e.g., relative to the medium) based on the data acquired by the localization sensor. The processing unit may also be configured to merge the data acquired by the probe with external data based on the data acquired by the localization sensor. Such external data may, for example, comprise medical data acquired previously, such as sonography, tomography, and/or MRI data. In this way, it becomes possible for the arm to scan a specific position in the medium or to move to a specific area of the medium during data acquisition in such a way that the data are acquired for the same area of the medium as that to which the external data refer.

Alternatively, the device 10 may comprise a user interface (for example, one or more sensors positioned at the level of the arm) configured to enable the user to control the movement of the arm. The user may, for instance, receive on the screen guiding information about the device intended to guide the probe's movement. This guiding information may be determined by the processing unit based on data acquired by the localization sensor and external data to enable a merging of the data acquired using the probe with the external data.

The localization sensor may use a radio triangulation technology and/or one or more “MEMS” (microelectromechanical systems), equipping the first arm and/or the probe holder.

Furthermore, the cable 202a may be extended outside of the backpack 110 and/or outside of the first arm 102a (and, for example, outside of the probe holder 102b). The device 10, in particular, its carrying system 100, may, for example, comprise one or more winders 102c configured for enabling the cable 202a to extend or unwind. Optionally, the device, in particular, the coil 102c, may be configured to retract an extended cable automatically.

As a result, since the cable on the probe may be extended and/or the first arm may be moved, the probe may be shifted by the user to any appropriate position during the data acquisition from a medium using the probe, for example.

Optionally, the device may be configured to comprise or be used with a number of probes. In this case, the device may, in turn, comprise multiple coils 102c. Furthermore, the probe holder may be configured to accommodate multiple probes. The processing unit 203 may also comprise multiple connectors 202b and/or the arm 102a may be configured to hold and/or to contain multiple cables 202a.

It is also noted that at least one probe or all of the probes may cooperate and/or communicate with the processing unit in a wireless manner, in other words without using cables. In this case, the probe holder 102b may be configured to hold the probes in a detachable manner when they are not [being used]. As a result, the user can grasp and detach a probe when they need the probe.

The first arm may be mobile in such a manner that the probe may be moved from the extended position to a retracted position in which the probe may be closer and/or adjacent to at least one element of the user's body, the backpack, and/or the belt system.

The medical device 10, in particular the medical equipment 200, may also comprise a screen device 201. It is also possible for the screen device to be an external element from the sonography equipment, and, for example, for the sonography equipment to comprise (solely) the probe 202. The screen device may be configured to display information based on the data processed by the treatment unit 203. For example, the screen device may be connected to the processing unit 203 via a cable and/or via a wireless communication interface. Furthermore, the screen device may be configured to display other information. Such additional information may, for example, comprise information relating to a patient (e.g., a patient file), the patient's medical data acquired in the past (e.g., sonography data and/or other data acquired using another device, such as magnetic resonance imaging (MRI) data and/or computerized tomography (CT) data).

The screen device may for example comprise a touchscreen. As a result, the screen device may be configured to enable the user to control the device 10 via the touchscreen. However, the device 10 may comprise any other user interface, for example, at the level of the belt system 103 (see for example FIG. 4).

The screen device 201 may be held by the second arm 101. In particular, the carrier system 100 may comprise the second arm 101. Such arm may be configured to hold the screen device 201 in an extended position relative to the user in such a way that the information displayed is visible to the user. For example, the second arm may be arranged in such a manner that it is extended above a shoulder and/or over the head 4 of the user 1.

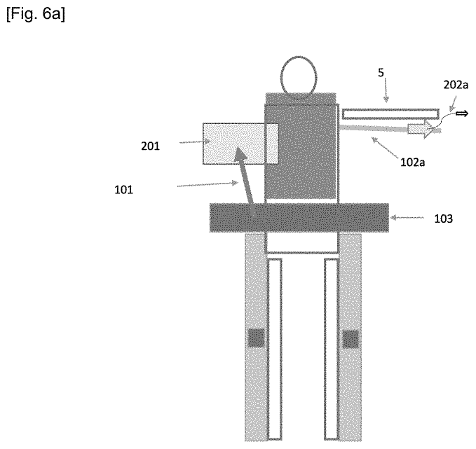

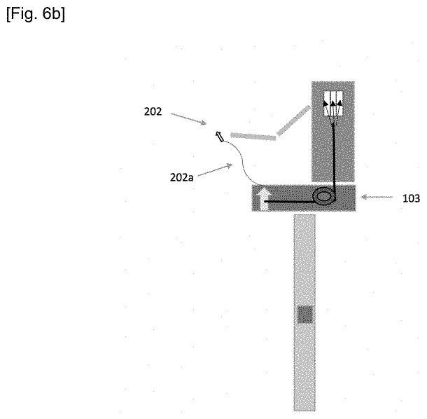

In general, the area A located in front of the user may be referred to as the intervention area (see FIG. 1b). The first and/or the second arm may extend towards this intervention area in order to enable the user to see the information on the screen and/or to use the equipment. The area B behind the user may be referred to as the storage area. The first and/or the second arm may be attached in or around the storage area. Some of the equipment may also be stored in or around the storage area, in particular, in the backpack. Alternatively, the first and/or the second arm may be attached to the belt and/or some of the equipment may be stored on the belt, as described, for instance, in the context of FIG. 6a. For example, the arm may be fixed to the backpack 110 and extend from this towards the front face of the user where the screen device 201 is held by the arm.

In one example, the first arm 102a extends above a shoulder of the user and the second arm 101 above the other shoulder. However, they may also extend above the same shoulder and/or over the head of the user 4.

The second arm 101 may be mobile in such a manner that the position of the screen device may be adjusted. As described above, the first and the second [arms] may be substantially similar in terms of their structure and/or material(s).

For example, the second arm may be shifted ensuring that the orientation and/or the position of the screen may be adjustable. The second arm may also be configured (e.g., by means of a joint) to enable the user to turn the screen device, for example, in such a way that a patient or other person who is in front of the user can see the information displayed on the screen.

The device 10, in particular, the carrier system 100, may also comprise one or two feet and/or legs 104 configured to support at least part of the weight of the carrier system 100 and/or of the medical equipment 200 on the ground where the user 1 stands. The single foot allows the user to assume a resting position and/or to reduce the sensation of carrying a load in certain configurations. The feet and/or legs may be configured to be linked to the user's lower limbs in order to follow the movement of the user's lower limbs. The foot or feet, or legs also ensure better stability of the device.

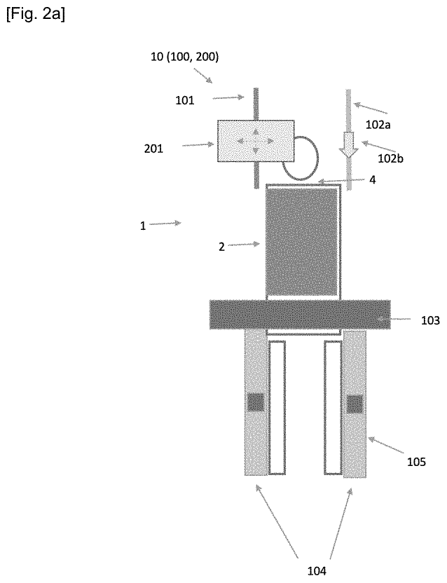

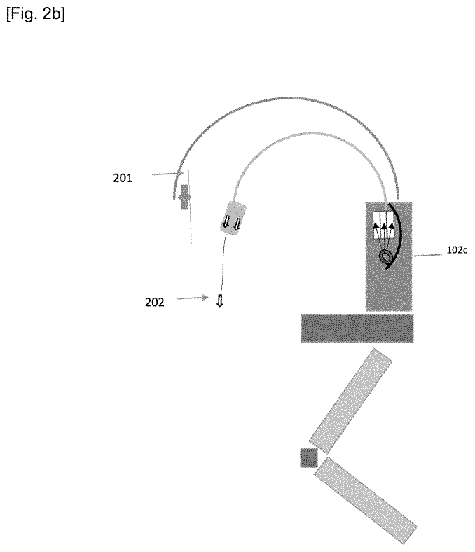

FIG. 2a is a schematic front view of the device in FIG. 1a and FIG. 1b in a seated position. FIG. 2b is a schematic lateral view of the device in FIG. 2a.

The embodiment according to the example shown in FIG. 2a and FIG. 2b may correspond to that of FIG. 1a and FIG. 1b. In particular, the embodiment according to the example of FIG. 2a and FIG. 2b may comprise the same elements and/or functions.

As specifically demonstrated in FIG. 2a, the feet and/or legs may each be configured to be articulated by the hinges 105. The feet and/or legs may also be configured to be mobile to enable the user to move and/or walk with their legs 3 when they are wearing the carrier system 100. As a result, the feet and/or legs may support at least some of the weight of the carrier system and/or the medical equipment on the ground when the user moves.

Furthermore, the feet and/or the legs may also be configured to allow a user to sit in a chair.

Furthermore, the feet and/or the legs may be configured to hold a seated and/or lowered position of the user without a chair. This position may be a working position (e.g., if the user is examining a patient who is sitting or lying down), decreasing the weight of the device on the user's legs and/or body. In particular, holding this working position enables the user to alleviate the strain caused by the constraints of the positions during the examination.

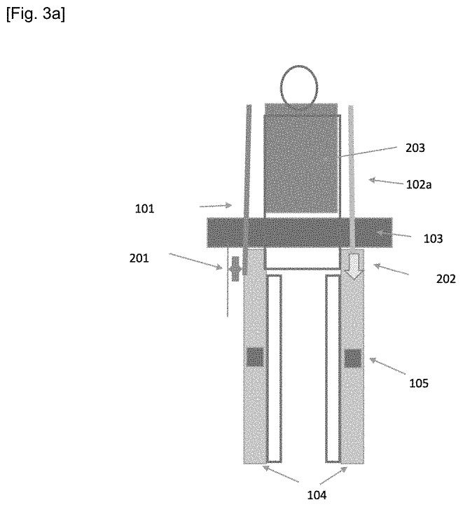

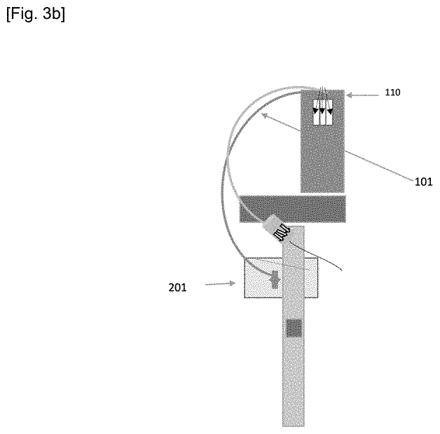

FIG. 3a is a schematic front view of the device in FIG. 1a, FIG. 1b, FIG. 2a, and FIG. 2b in a moving and/or stationary position. FIG. 3b is a schematic lateral view of the device in FIG. 3a.

The embodiment according to the example shown in FIG. 3a and FIG. 3b may correspond to that of FIG. 1a and FIG. 1b. In particular, the embodiment according to the example in FIG. 3a and FIG. 3b may comprise the same elements and/or functions. However, in the example illustrated, the elements may be in a retracted position in such a manner that the device 10 can be easily transported and/or stored/parked. This position may also be used if the user moves, for example, if they need to enter an intervention area during a catastrophe or accident, or in a hospital ward.

For example, the second arm 101 may be mobile in such a manner that the screen device 201 may be moved from the extended position to a retracted position in which the screen device may be closer and/or adjacent to at least one element of the user's body, the backpack, and the belt system.

Similarly, the first arm 102a may be mobile, enabling the probe 202 to be shifted from the extended position to a retracted position in which the probe holder and/or the probe 202 may be closer and/or adjacent to at least one element of the user's body, the backpack, and the belt system.

In the illustrated example, the first and the second arm may, in particular, be adjacent to the backpack 110 and the belt system 103. Optionally, the first and/or the second arm may comprise means of fastening configured to enable the fastening of the arms to the belt system and/or a leg 104, in particular, in a retracted position.

FIG. 4 is a schematic front view of the second embodiment of the device according to this disclosure, comprising a headset (for example, a head-mounted display or a virtual reality headset) and/or a user interface.

The embodiment according to the example demonstrated in FIG. 4 may correspond to that of FIG. 1a and FIG. 1b. In particular, the embodiment according to the example of FIG. 4 may comprise the same elements and/or functions. However, in this example the screen device and the second arm may be replaced by a head-mounted display. As a result, the user may wear the head-mounted display on their head. The head-mounted display may communicate with the processing unit 203 via a cable and/or wirelessly. Alternatively, it is also possible for the device 10 to comprise a combination of the screen device and the head-mounted display.

The backpack and/or belt system may be configured to carry the control unit 205 with a user interface. The control unit may enable the user to control the device 10 and/or the medical equipment 200. This may be useful, in particular, if the device 10 does not comprise a touchscreen device. The control unit 205 may, for instance, comprise one or more control buttons, switches, sliders, and potentially a trackpad.

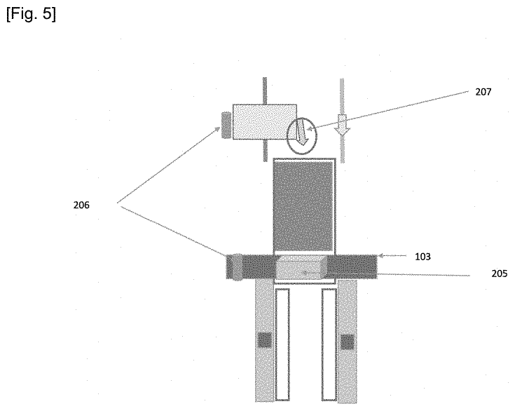

FIG. 5 is a schematic front view of the third embodiment of the device according to this disclosure, comprising accessories.

The embodiment according to the example shown in FIG. 5 may correspond to that of any of the other Figures. In particular, the embodiment according to the example in FIG. 5 may comprise the same elements and/or functions. However, in this example the device 10, in particular, the carrier system 100, may also comprise one or more accessories. The accessories may, for example, comprise a bottle of gel 206 which may be attached to the belt system and, in addition or alternatively, a bottle of gel 206 attached to the second arm 101 holding the screen device and/or to the first arm 102a holding the probe.

Furthermore, the accessories may comprise the light source 207 configured to illuminate an area located in front of the user, in particular, in such a manner that it illuminates the medium to be examined. The light source may be set in a mobile manner, ensuring that the illuminated area may be adjusted as needed, for instance, the position of the medium. For example, the light source may be attached to the first or second arm in a mobile manner. The device may also comprise a number of light sources, for example, attached to each of the two arms. The device may also comprise the control unit 205 as described, for example, in the context of FIG. 4. The control unit 205 may also be configured to control the accessories. For example, the control unit may include a button to turn the light source 207 on and off, and/or to control the intensity, the color, etc.

FIG. 6a is a schematic front view of the fourth embodiment of the device according to this disclosure. The embodiment according to the example shown in FIG. 6a may correspond to that of any of the other Figures. In particular, it may include the same elements and/or functions.

However, as the example in FIG. 6a demonstrates, the second arm 101 holding the screen device 201 may also be connected to the belt system 103 (instead of the backpack) and/or extend from it. For example, the second arm may be connected to the front or the side of a belt around the user's hips. A corresponding method of connection may also apply to the first arm 102a.

Furthermore, as the example in FIG. 6a shows, the first arm 102a may also be configured in such a way that it extends along the user's arm 5 and/or moves with the user. For example, it may also be connected to the backpack and/or extend from it in the area of the user's shoulder. In this example, the optional probe cable 202a may be guided along the first arm.

According to one example, the first arm 102a may be covered by a flexible sleeve that may surround the user's arm, for example, made of fabric, latex, or nitrile, and/or it may comprise an aseptic material that is either disposable or not. Such material may, for example, be configured to protect the user and the patient against infections. Moreover, the first arm may comprise and/or be covered by a flexible protection configured so that it also covers the probe and optionally the user's arm as well. Subsequently, the protection may also protect, on the one hand, the user's hand and the probe against infections and, on the other hand, the patient. In another example, the first arm 102a may comprise at least two rigid elements linked by a hinge. As a result, the first arm may be configured to support the weight of the probe and/or the probe cable. In yet another example, the second arm may simply comprise multiple straps (e.g., with Velcro or magnet fasteners) configured to be attached at one or more positions to the user's arm and to hold the probe and/or the probe cable. In particular, the cable may be located along and/or on the first arm 102a.

It is also possible for the second arm to comprise a glove and/or a sleeve surrounding the forearm configured to have a probe holder on the back of the user's hand. Said probe holder may also be configured to hold the probe in a position in such a way that the probe sensor extends in the direction of the forearm. As a result, the user may be able to acquire data using the probe by holding it (extending over the forearm) close to a medium.

According to one example, the probe may be set on the top of the wrist. It is also possible for the probe to be set in such a way that it rests in the user's hand. In this context, it is also possible for the probe to be set under the forearm and to be extendable/sliding towards the front, enabling the user to grasp it and “store” it easily as needed. The modifications to the first and to the second arm as described in the context of FIG. 6a may also be combined with the embodiments of the previous

Figures independently from one another (for example, just a change to the first arm or just a change to the second arm).

FIG. 6b is a schematic lateral view of the device in FIG. 6a with optional modifications.

The embodiment according to the example shown in FIG. 6b may correspond to that of any of FIG. 6a. In particular, it may comprise the same elements and/or functions.

However, as the example in FIG. 6b shows, the probe cable 202a may also extend from the belt system 103 (e.g., a belt around the user's hips) instead of extending along the first arm 102a. In this case, the probe cable may extend from a connector on the processing unit towards the belt unit, and inside the belt system towards an opening on the front side of the user. The cable may be extendable outside of the opening, for example, by means of one or more coils in the belt system.

It should be noted further that the second arm 101 and the screen device 201 are omitted from FIG. 6b simply for the reasons of simplifying the illustration. The embodiment according to the example in FIG. 6b may potentially include these elements.

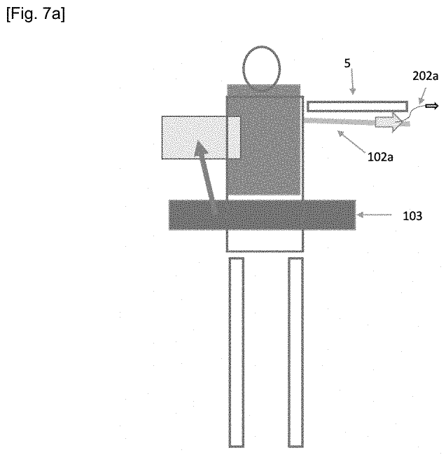

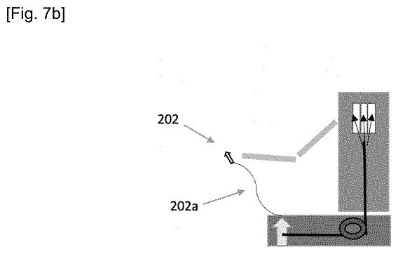

FIG. 7a is a schematic front view of the fifth embodiment of the device according to this disclosure. The embodiment according to the example shown in FIG. 7a may correspond to that of any of the other figures. In particular, it may comprise the same elements and/or functions.

However, as the example in FIG. 7a shows, the legs and/or the feet may be omitted. Instead, the carrier system 100 and, in particular, the belt system 103 may be configured to support the weight of the device 10, in particular, the medical equipment 200. As a result, the device may provide the user with better mobility.

In an example, the device 1 may be configured to be attached solely to the top of the user's body (e.g., from the user's hips and towards the top of their body).

In the illustrated example, the device may correspond to that of FIG. 6a, with the legs and/or feet omitted. Thanks to this configuration (described in the context of FIG. 6a), the elements of the device (in particular, the screen and the probe) may be placed close to the user's body. As a result, the user can support the weight of these elements with less effort and the omission of the legs and/or feet may thus be compensated for, at least in part.

FIG. 7b is a schematic lateral view of the device in FIG. 7a with optional modifications. In the illustrated example, the device may correspond to that of FIG. 6b, with the legs and/or feet omitted.

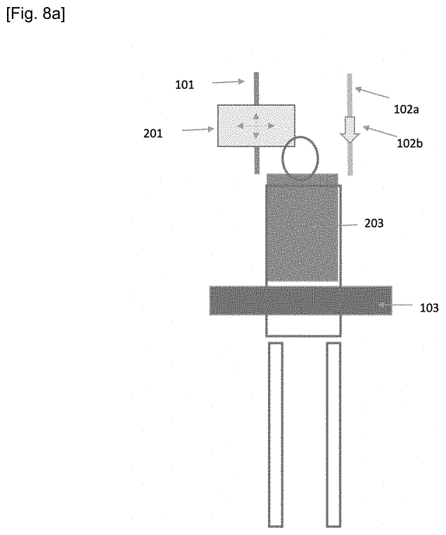

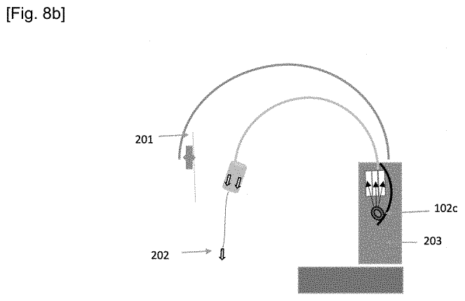

FIG. 8a is a schematic front view of the sixth embodiment of the device according to this disclosure. FIG. 8b is a schematic lateral view of the device in FIG. 8a. The embodiment according to the example shown in FIG. 8a may correspond to that of any of the other FIGS. 1a to 6b. In particular, it may comprise the same elements and/or functions.

However, as the example in FIG. 8a shows, the legs and/or the feet may be omitted. Instead, the carrier system 100 and, in particular, the belt system 103 may be configured to support the weight of the device 10, in particular, the medical equipment 200. As a result, the device may provide the user with better mobility.

In an example, the device 1 may be configured to be attached solely to the top of the user's body (e.g., from the user's hips and towards the top).

In the illustrated example, the device may correspond to that of FIG. 1a, with the legs and/or feet omitted. Thanks to this configuration (described in the context of FIG. 1a), the screen device and/or the probe (or the probe holder) may be arranged in a comfortable position for the user. In other words, the two elements may be placed facing the user. In particular, the screen device may be established in such a manner that the user can easily read the display on the screen, regardless of the user's position (e.g., standing, sitting, walking, changing position relative to a patient, etc.). Furthermore, the probe (or the probe holder) may also be arranged in such a manner that the user can easily reach and/or grasp it regardless of their position.

All of these embodiments and other examples as described above are given solely by way of a non-limiting example and may be combined and/or modified within the scope of the following claims.

A reference to a patent document or any other element identified as being prior art may not be considered to be an admission that the document or the other element was known or that the information it contains was part of general common knowledge at the priority date of any of the claims.

1. A portable medical device comprising:
a medical equipment for the acquisition of data and a carrier system, wherein the medical equipment comprises:
a processing unit configured to process the data acquired using an ultrasound probe associated with the processing unit, and
wherein the carrier system is configured to equip a body of a user of the device with the medical equipment.

2. The portable medical device according to claim 1, wherein at least one of:
the medical equipment further comprises the ultrasound probe and
the processing unit at least one of comprises a battery and is configured to be independently powered by an external electrical supply.

3. The portable medical device according to claim 1, further comprising a control unit with a user interface configured to enable the user to control the medical device.

4. The portable medical device according to claim 1, wherein the processing unit is configured to at least one of receive and process external data from an external system.

5. The portable medical device according to claim 4, further comprising at least one of a display screen and a head-mounted display,
wherein the processing unit is configured to ensure that the at least one of display screen and the head-mounted display displays information based on at least one of the processed data and the external data.

6. The portable medical device according to claim 1, wherein at least one of the carrier system is configured to be at least one of adjusted to the body of the user and installed on the body of the user and the carrier system comprises a backpack configured to carry the processing unit.

7. The portable medical device according to claim 6, wherein the backpack comprises a belt system configured to be worn on a torso of the user and to use the torso of the user to support at least some of the weight of at least one of the carrier system and the medical equipment.

8. The portable medical device according to claim 7, wherein at least one of the backpack and the belt system is configured to carry at least one of the battery and/or, the control unit and the accessories configured to be able to be used in at least one of preparation for and during a medical examination.

9. The portable medical device according to claim 1, wherein the carrier system comprises a first arm configured to hold the probe in a position allowing the probe to be grasped by a hand of the user.

10. The portable medical device according to claim 9, wherein the first arm comprises a probe holder configured to hold at least one of the probe and the first arm is further configured to at least one of hold and contain a cable connecting the probe to the processing unit.

11. The portable medical device according to claim 10, wherein the cable is extendable at least one of outside of the backpack and outside of the first arm.

12. The portable medical device according to claim 9, wherein at least one of the first arm a second arm and the probe holder is motorized, and the processing unit is configured to automatically control the movement of the at least one of the first arm, the second arm and the probe holder.

13. The portable medical device according to claim 10, wherein at least one of the first arm and the probe holder comprises a localization sensor and the processing unit is configured to at least one of:
determine a movement of the probe based on the data acquired by the localization sensors and
merge the data acquired by the probe with external data based on the data acquired by the localization sensor.

14. The portable ultrasound device according to claim 13, wherein the first arm is mobile in such a manner that at least one of:
the position adopted by the probe may be adjusted; and
the probe may be shifted from the extended position to a retracted position in which the probe is at least one of closer and adjacent to at least one element of at least one of the body of the user, the backpack and the belt system.

15. The portable medical device according to claim 14, wherein the carrier system comprises a second arm configured to hold the display screen in an extended position relative to the user, ensuring that the information displayed is accessible to at least one of the user and a patient.

16. The portable medical device according to claim 15, wherein:
the second arm is mobile in such a manner that at least one of:
the position of the display screen may be adjusted; and
the display screen may be shifted from the extended position to a retracted position in which the display screen is at least one of closer and adjacent to at least one element of the body of the user, the backpack, and the belt system.

17. The portable medical device according to claim 15, wherein at least one of:
at least one of the first and the second arm is attached in a mobile manner to at least one of the backpack and the belt system; and
at least one of the first and the second arm is configured to be held by the at least one of the backpack and the belt system.

18. The portable medical device according to claim 1, wherein:
the carrier system comprises at least one of a foot and a leg configured to support at least part of the weight of at least one of the carrier system and the medical device on the ground the user is situated on.

19. The portable medical device according to claim 18, wherein:
the at least one foot and leg is configured to be at least one of articulated and mobile in such a manner that the user can at least one of move and walk while the at least one of the foot and the leg supports at least some of the weight of at least one of the carrier system and the medical equipment on the ground.

20. The portable medical device according to claim 18, wherein:
the at least one foot and leg is configured to be connected to the user's lower limbs in order to follow the movement of the user's lower limbs.

21. The portable medical device according to claim 18, wherein:
the carrier system comprises an exoskeleton, wherein the exoskeleton comprises at least one of the following elements:
the backpack,
the belt system,
the first arm,
the second arm, and
the at least one foot and leg.
